(19) United States
(12) Patent Application Publication
(10) Pub. No.: US 2013/0059973 A1
(43) Pub. Date: Mar. 7, 2013

(54) POLYISOCYANATE PREPOLYMERS AND THEIR USE

(71) Applicants: Christian Wamprecht, Neuss (DE); Christos Karafilidis, Leverkusen (DE); Christoph Gürtler, Koln (DE); Beate Baumbach, Burscheid (DE)

(72) Inventors: Christian Wamprecht, Neuss (DE); Christos Karafilidis, Leverkusen (DE); Christoph Gürtler, Koln (DE); Beate Baumbach, Burscheid (DE)

(73) Assignee: BAYER INTELLECTUAL PROPERTY GMBH, Monheim (DE)

(21) Appl. No.: 13/696,221

(22) PCT Filed: May 2, 2011
(86) PCT No.: PCT/EP11/56954

§ 371 Date: Nov. 5, 2012

(30) Foreign Application Priority Data
May 6, 2010 (DE) 10 2010 019 504.9

(51) Int. Cl. C08G 18/72; C07C 265/14; C09J 175/08; C09D 175/08; C08G 18/76; C08G 18/75

(52) U.S. Cl. 524/590; 528/66; 558/275; 558/276

(57) ABSTRACT

The invention provides polyisocyanate prepolymers, characterised in that they contain polyether carbonate polyols as structural component, their preparation and their use as isocyanate component in 1- and 2-component systems for surface-coating compositions, adhesives and sealing materials.

The invention provides polyisocyanate prepolymers, characterised in that they contain polyether carbonate polyols as structural component, their preparation and their use as isocyanate component in 1- and 2-component systems for surface-coating compositions, adhesives and sealing materials.

Isocyanate-functional prepolymers are used in many technical fields, in particular for the adhesive bonding and coating of substrates as well as in sealing materials. Both moisture-curing 1-component systems and 2-component systems are used, polyols and/or polyamines frequently being used as reactants for the isocyanate-containing prepolymers.

Moisture-curing surface-coating compositions, adhesives and sealing materials and their preparation belong to the general prior art and are described many times in the literature. All isocyanate-group-containing prepolymers that are not stored with the absolute exclusion of moisture lose isocyanate groups over time by reaction with atmospheric moisture. Exposure to high temperatures encourages this process considerably. This reaction proceeds rapidly at the surface; diffusion into the inside of, for example, moulded articles, foams, surface-coating films or adhesive and sealing material layers can take a long time. As long as this reaction takes place, the molar mass, or crosslinking density, increases and the physical properties change accordingly.

In the field of surface-coating compositions and adhesives in particular, it is desirable for the reaction of the free isocyanate groups of the prepolymer with atmospheric moisture to take place as quickly and as completely as possible in order to obtain the finished use properties at an early stage. Nevertheless, the prepolymer must have very good stability to storage. In order to accelerate the curing process, external catalysts, such as, for example, organic tin compounds (dibutyltin dilaurate) or aminic accelerators (dimorpholino diethyl ether), are frequently added to the formulations. However, such catalysts can adversely affect the stability to storage with exposure to high temperatures, in particular of prepolymers based on reactive aromatic isocyanates, as well as the property profile of the adhesive. There has therefore been no lack of attempts to provide isocyanate-containing prepolymers of high reactivity which possess high reactivity towards moisture without the addition of external catalysts. Such prepolymers are frequently based on polyether or polyester polyols containing nitrogen atoms. These products are preferably used in one-component, moisture-curing foam applications.

DE OS 1922626 and EP-A 796 880 describe processes for the preparation of polyurethane-based one-component systems which are stable to storage and dry quickly with atmospheric moisture. They are solvent- and plasticiser-containing formulations which can be used as binders in one-component paint systems.

Moisture-curing adhesive compositions are described, for example, in WO-A 95/10555, DE OS 102 37 649, DE OS 103 04 153, EP-A 1 072 620, WO-A 00/44803, WO-A 2009000405. In order to accelerate curing, in some cases amine-containing polyethers as structural component or catalysts based on morpholine derivatives are used.

There is a constant need for alternative or improved isocyanate-functional prepolymers for the above uses. As binders in adhesives, coating compositions or sealing materials, the prepolymers are to fulfil the demands made of the particular application-related properties as well as possible.

Accordingly, it was an object of the present invention to provide isocyanate-functional prepolymers having improved properties, in particular more rapid drying when used as an adhesive, sealing material or in surface-coating applications.

Surprisingly, it has been found that this object is achieved by isocyanate-functional prepolymers which contain polyether carbonate polyols as structural component. The invention further provides isocyanate-functional prepolymers, the preparation thereof, and their use in adhesives, sealing materials and in surface-coating applications.

The polyether carbonate polyol preferably has a mean OH functionality (mean number of OH groups per molecule) of from 2 to 6, preferably from 2 to 4, particularly preferably from 2 to 3 and most particularly preferably 2.

The polyether carbonate polyol preferably has a content of carbonate groups (calculated as CO₂) of at least 1 wt. %, preferably of at least 5 wt. %, particularly preferably of at least 10 wt. % and most particularly preferably of from 15 to 30 wt. %.

The polyether carbonate polyol preferably has a number-average molecular weight of from 500 to 10,000, preferably from 500 to 5000, particularly preferably from 750 to 4000 and most particularly preferably from 1000 to 3500, measured by means of GPC (gel permeation chromatography).

Suitable polyether carbonate polyols are obtainable, for example, by addition of carbon dioxide and alkylene oxides to H-functional starter substances using multimetal cyanide catalysts, which are also referred to as DMC catalysts, for example according to WO 2008/013731. Although that specification mentions the possibility of using polyether carbonates generally in polyurethane foams, elastomers, coatings, sealing materials and adhesives, no information is given about expected effects as regards the properties of the resulting products.

The preparation of polyether carbonate polyols is conventionally carried out by catalytic addition of alkylene oxides and carbon dioxide to H-functional starter substances.

There can be used as alkylene oxides pure alkylene oxides, mixtures of alkylene oxides or mixtures of oxides of commercially available raffinate streams. In general, alkylene oxides having from 2 to 24 carbon atoms can be used for the process according to the invention. Examples which may be mentioned include ethylene oxide, propylene oxide, 1-butene oxide, 2,3-butene oxide, 1-pentene oxide, 1-hexene oxide, 1-octene oxide, 1-decene oxide, butadiene monoxide, isoprene monoxide, cyclopentene oxide, cyclohexene oxide, styrene oxide and mesitylene oxide. There are used in particular ethylene oxide, propylene oxide and styrene oxide, particularly preferably propylene oxide and styrene oxide and most particularly preferably propylene oxide.

For the preparation of the polyether carbonate polyols used according to the invention, alkylene oxides and carbon dioxide are added to H-functional starter substances. Suitable starter substances which can be used are all compounds having H atoms active for the alkoxylation. Groups active for the alkoxylation having active H atoms are —OH, —NH, —SH and —CO₂H, preferably —OH and —NH and particularly preferably —OH.

Suitable starter substances which can be used are, for example, water, polyhydric alcohols, polyvalent amines, polyvalent thiols, polyhydric aminoalcohols, polyhydric thioalcohols, polyether polyols, polyester polyols, polyester ether polyols, polycarbonate polyols, polyethyleneimines, polyether amines (e.g. so-called Jeffamine® from Huntsman, such as, for example, D-230, D-400, D-2000, T-403, T-3000, T-5000 or corresponding products from BASF, such as, for example, polyether amine D230, D400, D200, T403, T5000), polytetrahydrofurans (e.g. PolyTHF® from BASF, such as, for example, PolyTHF® 250, 650S, 1000, 10005, 1400, 1800, 2000), polytetrahydrofuranamines (BASF product polytetrahydrofuranamine 1700), polyether thiols and polyacrylate polyols. In a particular embodiment there can be used as starter substances castor oil, the mono- or di-glyceride of ricinoleic acid, or monoglycerides of fatty acids. Furthermore, chemically modified mono-, di- and/or tri-glycerides of fatty acids or C₁-C₂₄-alkyl fatty acid esters can be used, into which there are introduced chemically on average at least 2 OH groups per molecule. Examples which may be mentioned in this context include commercial products such as Lupranol Balance® (BASF AG), Merginol® types (Hobum Oleochemicals GmbH), Sovermol® types (Cognis Deutschland GmbH & Co. KG) and Soyol™ types (USSC Co.).

All the mentioned substances are used as starter substances either as individual substances or as mixtures of at least 2 of the mentioned compounds.

Polyhydric alcohols suitable as starter substances are, for example, dihydric alcohols, such as ethylene glycol, diethylene glycol, propylene glycol, dipropylene glycol, 1,3-propanediol, 1,4-butanediol, 1,4-butenediol, 1,4-butynediol, neopentyl glycol, 1,5-pentanediol, methylpentanediols, such as, for example, 3-methyl-1,5-pentanediol, 1,6-hexanediol; 1,8-octanediol, 1,10-decanediol, 1,12-dodecanediol, bis-(hydroxymethyl)-cyclohexanes, such as 1,4-bis-(hydroxymethyl)cyclohexane, hydroquinone bis(2-hydroxyethyl) ether, diethylene glycol, triethylene glycol, tetraethylene glycol, polyethylene glycols, such as, for example, polyethylene glycol 400, dipropylene glycol, tripropylene glycol, polypropylene glycols, dibutylene glycol and polybutylene glycols; trihydric alcohols, such as, for example, trimethylolpropane, glycerol, trishydroxyethyl isocyanurate, castor oil; tetrahydric alcohols, such as, for example, pentaerythritol; polyalcohols, such as, for example, sorbitol, hexitol, sucrose, starch, starch hydrolysates, cellulose, cellulose hydrolysates, hydroxy-functionalised fats and oils, in particular castor oil. All modification products of these mentioned alcohols with varying amounts of ε-caprolactone are likewise suitable as starter substances. There come into consideration as dihydric alcohols in particular ethylene glycol, propylene glycol, 1,3-propanediol, 1,3-butanediol, 1,4-butanediol, 1,5-pentanediol, 2-methyl-1,3-propanediol, neopentyl glycol, 1,6-hexanediol. Preference is given to alcohols of the general formula HO(CH₂)x-OH, wherein x is a number from 1 to 20, preferably an even number from 2 to 20. Examples thereof are ethylene glycol, 1,4-butanediol, 1,6-hexanediol, 1,8-octanediol and dodecane-1,12-diol. Neopentyl glycol is also preferred.

As starter substances for the preparation according to the invention of polyether carbonate polyols there are used in particular alcohols having functionalities of from 2 to 6, either as an individual substance or as a mixture of at least 2 of the mentioned alcohols. Di- and/or tri-functional alcohols are preferably used as starter substances.

The starter substances can also be selected from the substance class of the polyether polyols, in particular those having a molecular weight Mn in the range from 100 to 4000 g/mol (GPC). Polyether polyols having a functionality of at least 2, preferably from 2 to 6, particularly preferably from 2 to 4 are used as the polyether polyols. Preference is given to polyether polyols that are composed of repeating ethylene oxide and propylene oxide units, preferably having a content of from 35 to 100% propylene oxide units, particularly preferably having a content of from 50 to 100% propylene oxide units. These can be random copolymers, gradient copolymers, alternating or block copolymers of ethylene oxide and propylene oxide. Suitable polyether polyols composed of repeating propylene oxide and/or ethylene oxide units are, for example, the Desmophen®, Acclaim®, Arcol®, Baycoll®, Bayfill®, Bayflex®, Baygal®, PET® and polyether polyols from Bayer MaterialScience AG, such as, for example, Desmophen® 3600Z, Desmophen® 1900U, Acclaim® Polyol 2200, Acclaim® Polyol 40001, Arcol® Polyol 1004, Arcol® Polyol 1010, Arcol® Polyol 1030, Arcol® Polyol 1070, Baycoll® BD 1110, Bayfill® VPPU 0789, Baygal® K55, PET® 1004, Polyether® 5180. Further suitable homo-polyethylene oxides are, for example, the Pluriol® E brands from BASF AG, suitable homo-polypropylene oxides are, for example, the Pluriol® P brands from BASF AG, suitable mixed copolymers of ethylene oxide and propylene oxide are, for example, the Pluronic® PE or Pluriol® RPE brands from BASF AG.

The starter substances can also be selected from the substance class of the polyester polyols, in particular those having a molecular weight Mn in the range from 200 to 4500 g/mol (GPC). At least difunctional polyesters are used as polyester polyols. Polyester polyols preferably consist of alternating acid and alcohol units. There are used as acid components, for example, succinic acid, maleic acid, adipic acid, phthalic anhydride, phthalic acid, isophthalic acid, terephthalic acid or mixtures of the mentioned acids and/or anhydrides. There are used as alcohol components, for example, ethanediol, 1,2-propanediol, 1,3-propanediol, 1,4-butanediol, 1,5-pentanediol, neopentyl glycol, 1,6-hexanediol, 1,4-bis-(hydroxymethyl)-cyclohexane, diethylene glycol, dipropylene glycol or mixtures of the mentioned alcohols. If divalent or polyvalent polyether polyols are used as the alcohol component, polyester ether polyols which can likewise be used as starter substances for the preparation of the polyether carbonate polyols are obtained. Preferably, polyether polyols with Mn=from 150 to 2000 g/mol (GPC) are used for the preparation of the polyester ether polyols.

Polycarbonate diols can further be used as starter substances, in particular polycarbonate diols having a molecular weight Mn in the range from 150 to 4500 g/mol (GPC), which are prepared, for example, by reaction of phosgene, dimethyl carbonate, diethyl carbonate or diphenyl carbonate and difunctional alcohols or polyester polyols or polyether polyols. Examples of polycarbonates are to be found, for example, in EP-A 1359177. For example, there can be used as polycarbonate diols the Desmophen® C types from Bayer MaterialScience AG, such as, for example, Desmophen® C 1100 or Desmophen® C 2200.

In a further embodiment of the invention, polyether carbonate polyols can be used as starter substances. In particular, polyether carbonate polyols according to the process described herein are used. These polyether carbonate polyols used as starter substances are prepared beforehand in a separate reaction step.

There are preferably used as H-functional starter substances water, diethylene glycol, dipropylene glycol, glycerol, trimethylolpropane, pentaerythritol, castor oil, sorbitol and polyether polyols composed of repeating polyalkylene oxide units. Particular preference is given to diethylene glycol, dipropylene glycol, glycerol, trimethylolpropane, polyether polyols composed of propylene oxide or of propylene oxide and ethylene oxide and having a functionality of from 2 to 3. The polyether polyols preferably have a molecular weight Mn in the range from 62 to 4500 g/mol (GPC) and a functionality of from 2 to 4, and in particular a molecular weight Mn in the range from 62 to 3000 g/mol (GPC) and a functionality of from 2 to 3. The preferred starter substances are used either as an individual substance or as a mixture of at least 2 of the mentioned substances. The preparation of the polyether carbonate polyols is carried out by catalytic addition of carbon dioxide and alkylene oxides to starter substances having H atoms active for the alkoxylation.

The double metal cyanide catalysts used for the preparation of the polyether carbonate polyols preferably have the general formula (IV) M1a[M2(CN)b(A)c]d·fM1gXn·h(H2O)·eL (IV) wherein M1 denotes a metal ion selected from the group containing Zn²⁺.Fe²⁺, Co³⁺, Ni²⁺, Mn²⁺, Co²⁺, Sn²⁺, Pb²⁺, Mo⁴⁺, Mo⁶⁺, Al³⁺, V⁴⁺, V⁵⁺, Sr²⁺, W⁴⁺, W⁶⁺, Cr²⁺, Cr³⁺, Cd²⁺, M2 denotes a metal ion selected from the group containing Fe²⁺, Fe³⁺, Co²⁺, Co³⁺, Mn²⁺, Mn³⁺, V⁴⁺, V⁵⁺, Cr²⁺, Cr³⁺, Rh³⁺, Ru²⁺, Ir³⁺and M1 and M2 are the same or different, A denotes an anion selected from the group containing halide, hydroxide, sulfate, carbonate, cyanide, thiocyanate, isocyanate, cyanate, carboxylate, oxalate and nitrate, X denotes an anion selected from the group containing halide, hydroxide, sulfate, carbonate, cyanide, thiocyanate, isocyanate, cyanate, carboxylate, oxalate and nitrate, L denotes a water-miscible ligand selected from the group containing alcohols, aldehydes, ketones, ethers, polyethers, esters, ureas, amides, nitriles and sulfides, and a, b, c, d, g and n are so chosen that the electroneutrality of the compound is ensured, and e denotes the coordination number of the ligand, f denotes a fractional number or an integer greater than or equal to 0, h denotes a fractional number or an integer greater than or equal to 0.

The DMC catalysts suitable for the preparation of the polyether carbonates are known in principle from the prior art (see e.g. U.S. Pat. No. 3,404,109, U.S. Pat. No. 3,829,505, U.S. Pat. No. 3,941,849 and U.S. Pat. No. 5,158,922). Preference is given to the use of improved, highly active DMC catalysts, which are described, for example, in U.S. Pat. No. 5,470,813, EP-A 700 949, EP-A 743 093, EP-A 761 708, WO 97/40086, WO 98/16310 and WO 00/47649. These catalysts have extraordinarily high activity and permit the preparation of polyether polyols at very low catalyst concentrations (25 ppm or less), so that separation of the catalyst from the finished product is generally no longer required. A typical example are the highly active DMC catalysts described in EP-A 700 949, which contain, in addition to a double metal cyanide compound (e.g. zinc hexacyanocobaltate(III)) and an organic complex ligand (e.g. tert-butanol), also a polyether having a number-average molecular weight greater than 500 g/mol (GPC).

The catalyst is in most cases used in an amount of less than 1 wt. %, preferably in an amount of less than 0.5 wt. %, particularly preferably in an amount of less than 500 ppm and in particular in an amount of less than 100 ppm, in each case based on the weight of the polyether carbonate polyol.

The preparation of the polyether carbonate polyols is carried out in a pressurised reactor. The metered addition of one or more alkylene oxides and of the carbon dioxide takes place after optional drying of a starter substance or of the mixture of a plurality of starter substances and the addition of the DMC catalyst and the additive(s), which are added before or after the drying in solid form or in the form of a suspension. The metered addition of one or more alkylene oxides and of the carbon dioxide can in principle be carried out in various ways. The start of the metered addition can take place from the vacuum or at a previously chosen preliminary pressure. The preliminary pressure is preferably established by passing in an inert gas such as, for example, nitrogen, the pressure being set at from 10 mbar to 5 bar, preferably from 100 mbar to 3 bar and more preferably from 500 mbar to 2 bar.

The metered addition of one or more alkylene oxides and of the carbon dioxide can take place simultaneously or sequentially, it being possible for the entire amount of carbon dioxide to be added at once or in a metered manner over the reaction time. A metered addition of the carbon dioxide is preferably carried out. The metered addition of one or more alkylene oxides takes place simultaneously or sequentially with the metered addition of the carbon dioxide. If a plurality of alkylene oxides are used in the synthesis of the polyether carbonate polyols, then the metered addition thereof can take place simultaneously or sequentially via separate metered additions or via one or more metered additions, at least two alkylene oxides being metered in as a mixture. Via the nature of the metered addition of the alkylene oxides and of the carbon dioxide it is possible to synthesise random, alternating, block-like or gradient-like polyether carbonate polyols.

Preferably, an excess of carbon dioxide is used, in particular the amount of carbon dioxide is determined via the total pressure under reaction conditions. An excess of carbon dioxide is advantageous due to the slowness of carbon dioxide to react. It has been shown that the reaction at from 60 to 150° C., preferably at from 70 to 140° C., particularly preferably at from 80 to 130° C., and pressures from 0 to 100 bar, preferably from 1 to 90 bar and particularly preferably from 3 to 80 bar, produces the polyether carbonate polyols. At temperatures below 60° C., the reaction comes to a halt. At temperatures above 150° C., the amount of undesirable secondary products increases considerably.

The polyether carbonates are the structural component for the isocyanate-functional prepolymers according to the invention. The polyether carbonates are used either on their own or in combination with other polyol components. The other polyol components include polyether polyols, polyester polyols, polycarbonate polyols, polyether ester polyols and other polyols as have already been mentioned above in the description of the starter substances for the polyether carbonate polyols. However, in the preparation of the isocyanate-functional prepolymers according to the invention, higher molar weights are possible for the other polyol components than those mentioned above. Polyether polyols that are highly suitable also include those which contain tertiary amino groups. Such tertiary amino groups can be incorporated by suitably choosing the starter component in the preparation of the polyethers. There are suitable, for example, ethylenediamine, hexamethylenediamine, isophoronediamine, 4,4′-diaminodicyclohexylmethane, triethanolamine, 2,3-diaminotoluene, 2,4-diaminotoluene, etc. As internal catalysts, these tertiary amino groups increase the reactivity of the isocyanate-functional prepolymers towards moisture and other reactants, such as, for example, polyols.

There come into consideration as the isocyanate component for the preparation of the isocyanate-containing prepolymers according to the invention the conventional, commercially available aliphatic, araliphatic and aromatic di- and poly-isocyanates. These include monomeric and polymeric isocyanates having isocyanate functionalities of on average at least 2, preferably from 2 to 6, particularly preferably from 2 to 5 and most particularly preferably from 2 to 4. Mention may be made in particular of monomeric diisocyanates (functionality=2), for example aliphatic isocyanates, such as, for example, 1,4-butane diisocyanate, hexamethylene diisocyanate, trimethylhexamethylene diisocyanate and triisocyanatononane; cycloaliphatic isocyanates, such as, for example, isophorone diisocyanate, 4,4′-dicyclohexylmethane diisocyanate and 1,4-diisocyanato-cyclohexane; araliphatic isocyanates, such as, for example, p-xylylene diisocyanate and tetramethylxylylene diisocyanate; and aromatic isocyanates, such as, for example, 2,4-diisocyanatotoluene, 2,6-diisocyanatotoluene and mixtures of these isomers, 4,4′-diisocyanatodiphenylmethane, 2,4′-diisocyanatodiphenylmethane and mixtures of these isomers. Also highly suitable are polyisocyanates based on these mentioned monomeric diisocyanates having isocyanate functionalities >2. Such polyisocyanates are generally free of monomeric diisocyanates or contain them in only very small amounts of <1 wt. %, preferably <0.5 wt. % and particularly preferably <0.3 wt. %. Such polyisocyanates are isocyanate-functional compounds having urethane groups, biuret groups, isocyanurate groups, iminooxadiazinedione groups, uretdione groups and/or allophanate groups.

In the preparation of the isocyanate-functional prepolymers according to the invention, the polyisocyanate components in question are conventionally placed in a reaction vessel in a molar excess, and the polyol components are metered in, either as a mixture or in succession, at temperatures in the range from 20 to 160° C., preferably from 40 to 140° C. Any heat of reaction that occurs is advantageously taken up by cooling so that the reaction between the isocyanate groups of the isocyanate components and the hydroxyl groups of the hydroxyl components proceeds at constant temperature. The reaction is finished when the desired isocyanate contents, or viscosities, of the isocyanate-functional prepolymers according to the invention have been reached. In the case of the use of monomeric diisocyanates, any residual amounts of those isocyanates that are present must be removed following the urethane reaction, for example by distillation or extraction, in order to obtain products having residual monomer contents of <1 wt. %, preferably <0.5 wt. % and particularly preferably <0.3 wt. %. In the case of the use of polyisocyanates for the preparation of the prepolymers according to the invention, the removal of excess residual monomers after the urethane reaction is not necessary because polyisocyanates already have residual monomer contents in the required range of <0.5 wt. %.

The reaction components are preferably used in relative proportions such that the above-described properties of the isocyanate-functional prepolymers, in particular the viscosity, the isocyanate content and the functionality, are achieved.

The resulting isocyanate-functional prepolymers according to the invention are suitable without further additives for use as one-component moisture-curing coatings, adhesives and sealing materials. The isocyanate-functional prepolymers according to the invention are further suitable for use as two-component curing coatings, adhesives and sealing materials. To that end, commercially available polyols and/or polyamines are used as reactants. Such polyols and/or polyamines have already been described above. Further polyols are additionally solvent-free and solvent-containing polyacrylate polyols, as are obtainable, for example, under the trade name Desmophen® A from Viverso GmbH, Bitterfeld. Aspartic acid esters can further be used as reactants for the isocyanate-functional prepolymers according to the invention. This particular type of polyamines are products having reduced reactivity of the secondary amino groups. As a result, it is possible to formulate two-component systems with an appropriate pot life in the range of from 10 to 60 minutes, which is not possible owing to the high reactivity of conventional compounds containing primary or secondary amino groups. Examples of suitable aspartic acid esters are Desmophen® NH 1220, Desmophen® NH 1420, Desmophen® NH 1520 and Desmophen® NH 1521 from Bayer MaterialScience AG.

The choice of suitable polyols and/or polyamines and of the isocyanate-functional prepolymers according to the invention is generally made so that the optimum product properties for the intended use are obtained.

### EXAMPLES

### Example 1

A mixture of 106.56 g of a polyether carbonate diol based on propylene oxide, carbon dioxide and 1,8-octanediol having a content of 24.3 wt. % incorporated carbon dioxide and an OH number of 60.8 mg KOH/g and 106.56 g of a polypropylene oxide polyether based on 1,2-diaminoethane having an OH number of 60 mg KOH/g is placed in a 1-litre four-necked flask and stirred for 1 hour at 120° C. under a vacuum of 20 mbar. The mixture is then cooled to 70° C. The resulting polyol mixture is metered in the course of about 30 minutes into a mixture of 200.32 g of a polyisocyanate based on diphenylmethane diisocyanate (MDI) having an NCO content of 31.5 wt. %, a content of 2,2′-MDI of 2.3%, a content of 2,4′-MDI of 12.6% and a content of 4,4′-MDI of 42.4% and a viscosity of 90 mPas at 25° C., and 85.96 g of a polyisocyanate based on MDI having an NCO content of 32.5 wt. %, a content of 2,4′-MDI of 32.2%, a content of 4,4′-MDI of 49.9% and a content of 2,2′-MDI of 7.3% and a viscosity of 21 mPas (25° C.). Heating is then carried out to 80° C. using an exothermic reaction which may occur. Stirring is carried out at 80° C. until the isocyanate content is constant. Then 0.3 g of isophthaloyl chloride in 0.3 g of a polyisocyanate based on MDI having an NCO content of 33.5%, a content of 2,4′-MDI of 60.0%, a content of 4,4′-MDI of 38.5% and a content of 2,2′-MDI of 0.8% and a viscosity of 12 mPas (25° C.) is added. There is obtained a brownish coloured polyisocyanate mixture having an NCO content of 16.2 wt. %, a viscosity of 7290 mPas (23° C.) and an average isocyanate functionality of about 2.8.

### Example 2

238 g of a polyether carbonate diol based on propylene oxide, carbon dioxide and 1,8-octanediol having a content of 24.3 wt. % incorporated carbon dioxide and an OH number of 60.8 mg KOH/g are placed in a 1-litre four-necked flask and stirred for 1 hour at 120° C. under a vacuum of 20 mbar. The mixture is then cooled to 70° C. The resulting polyol is metered in the course of about 30 minutes into 262 g of a polyisocyanate based on MDI having an NCO content of 33.5 wt. %, a content of 2,4′-MDI of 60.0%, a content of 4,4′-MDI of 38.5% and a content of 2,2′-MDI of 0.8% and a viscosity of 12 mPas (25° C.). Heating is then carried out to 80° C. using an exothermic reaction which may occur. Stirring is carried out at 80° C. until the isocyanate content is constant. There is obtained a brownish coloured polyisocyanate mixture having an NCO content of 15.4 wt. %, a viscosity of 980 mPas (23° C.) and an average isocyanate functionality of about 2.0.

### Example 3

### Comparison

A mixture of 107.25 g of a polyether diol based on propylene oxide having an OH number of 56 mg KOH/g and 107.25 g of a polypropylene oxide polyether based on 1,2-diaminoethane having an OH number of 60 mg KOH/g is placed in a 1-litre four-necked flask and stirred for 1 hour at 120° C. under a vacuum of 20 mbar. The mixture is then cooled to 70° C. The resulting polyol mixture is metered in the course of about 30 minutes into a mixture of 199.38 g of a polyisocyanate based on diphenylmethane diisocyanate (MDI) having an NCO content of 31.5 wt. %, a content of 2,2′-MDI of 2.3%, a content of 2,4′-MDI of 12.6% and a content of 4,4′-MDI of 42.4% and a viscosity of 90 mPas at 25° C., and 85.54 g of a polyisocyanate based on MDI having an NCO content of 32.5 wt. %, a content of 2,4′-MDI of 32.2%, a content of 4,4′-MDI of 49.9% and a content of 2,2′-MDI of 7.3% and a viscosity of 21 mPas (25° C.). Heating is then carried out to 80° C. using an exothermic reaction which may occur. Stirring is carried out at 80° C. until the isocyanate content is constant. Then 0.29 g of isophthaloyl chloride in 0.29 g of a polyisocyanate based on MDI having an NCO content of 33.5%, a content of 2,4′-MDI of 60.0%, a content of 4,4′-MDI of 38.5% and a content of 2,2′-MDI of 0.8% and a viscosity of 12 mPas (25° C.) is added. There is obtained a brownish coloured polyisocyanate mixture having an NCO content of 16.2 wt. %, a viscosity of 7320 mPas (23° C.) and an average isocyanate functionality of about 2.8.

### Example 4

### Comparison

240 g of a polyether diol based on propylene oxide having an OH number of 56 mg KOH/g are placed in a 1-litre four-necked flask and stirred for 1 hour at 120° C. under a vacuum of 20 mbar. The mixture is then cooled to 70° C. The resulting polyol is metered in the course of about 30 minutes into 260 g of a polyisocyanate based on MDI having an NCO content of 33.5%, a content of 2,4′-MDI of 60.0%, a content of 4,4′-MDI of 38.5% and a content of 2,2′-MDI of 0.8% and a viscosity of 12 mPas (25° C.). Heating is then carried out to 80° C. using an exothermic reaction which may occur. Stirring is carried out at 80° C. until the isocyanate content is constant. There is obtained a brownish coloured polyisocyanate mixture having an NCO content of 15.5 wt. %, a viscosity of 965 mPas (23° C.) and an average isocyanate functionality of about 2.0.

### Application-Related Tests:

### 1. Test for Reactivity as a Reactive Adhesive

In order to compare the reactivity, the dry-hard time (FBZ) and set-to-touch time (FTZ) according to ASTM D 5895 in the linear drying-recorder and the viscosity at 25° C. (rotary viscometer with coaxial cylinders, DIN 53019) were measured. The stability to storage at 70° C. was also measured in the form of the increase in viscosity over time. The polyisocyanate mixture is considered to be stable to storage when the viscosity has less than doubled in the course of 14 days' storage at 70° C.

| | | | | Stable to |
|---|---|---|---|---|
| | Viscosity | | | storage in the |
| | [mPas] | FBZ | FTZ | course of 14 d |
| Example No. | at 23° C. | [min] | [min] | at 70° C. |
| 1 | 7290 | 47 | 78 | Yes |
| 3 (comparison) | 7320 | 65 | 105 | Yes |

The polyisocyanate mixture according to the invention of Example 1 has a viscosity and stability to storage comparable with those of Example 3 (comparison). However, the reactivity of the polyisocyanate mixture according to the invention of Example 1, which is reflected in short dry-hard and set-to-touch times, is markedly higher than the reactivity of the comparison example.

### 2. Testing as a Coating Composition

100 g of each of the prepolymers of Examples 2 and 4 were combined at room temperature with a mixture of 63.5 g of Jeffamin® SD 2001 (Huntsman, USA) and 23.5 g of Ethacure® 100 (DETDA, Albemarle, USA) (NCO/NH=1.1: 1.0). Corresponding films were then applied to a glass sheet by means of a 150 μm doctor blade. The properties of the coatings are summarised in Table 2.

| Prepolymer example | 2 | 4 (comparison) |
|---|---|---|
| Non-tacky film after [min] | 3 | 4 |
| Shore hardness D: DIN 53505 | | |
| After 1 d | 48 | 46 |
| After 2 d | 53 | 50 |
| After 7 d | 55 | 52 |
| Elongation at break ISO EN 527 [%] | 418 | 420 |
| Tensile strength ISO EN 527 [MPa] | 18.9 | 18.5 |

Polyisocyanate 2 according to the invention based on a polyether carbonate polyol yields, in combination with polyamines, a coating which dries very quickly and has high hardness, good elongation at break and high tensile strength. Polyisocyanate 4 not according to the invention, based on a carbonate-group-free polyether, yields, in combination with polyamines, a coating which likewise has comparably good elongation at break and tensile strength, but the drying time until a non-tacky film is obtained is longer and the film hardness is lower than in the case of the polyisocyanate according to the invention of Example 2.

1.-14. (canceled)

15. A polyisocyanate mixture, comprising a polyether carbonate polyol.

16. The polyisocyanate mixture according to claim 15, characterised in that the polyether carbonate polyol is obtainable by addition of carbon dioxide and alkylene oxides to H-functional starter substances using multimetal cyanide catalysts (DMC catalysts).

17. The polyisocyanate mixture according to claim 15, characterised in that it contains only aromatic isocyanates.

18. The polyisocyanate mixture according to claim 15, characterised in that it contains only aliphatic isocyanates.

19. The polyisocyanate mixture according to claim 15, characterised in that it contains only cycloaliphatic isocyanates.

20. The polyisocyanate mixture according to claim 15, characterised in that it contains mixtures of aromatic and aliphatic isocyanates.

21. The polyisocyanate mixture according to claim 15, characterised in that it contains mixtures of aromatic and cycloaliphatic isocyanates.

22. The polyisocyanate mixture according to claim 15, characterised in that it contains mixtures of aliphatic and cycloaliphatic isocyanates.

23. The polyisocyanate mixture according to claim 15, characterised in that it has an isocyanate content of from 3 to 30 wt. % and isocyanate functionality of

24. The polyisocyanate mixture according to claim 15, characterised in that it has an isocyanate content of from 5 to 25 wt. % and isocyanate functionality of 2.

25. A coating composition, surface-coating composition, adhesive, or sealing material comprising the polyisocyanate mixture according to claim 15.

26. A one-component moisture-curing coating, curing coating, adhesive or sealing material comprising the polyisocyanate mixture according to claim 15 without further additives.

27. A two-component curing coating, adhesive or sealing material obtainable by reaction of
(i) the polyisocyanate mixture according to claim 15 with
(ii) at least one component selected from the group of the polyols and polyamines.

28. The two-component curing coating, adhesive or sealing material according to claim 27, characterised in that polyacrylate polyols or aspartic acid esters are used as component (ii).
